(19) United States
(12) Plant Patent Application Publication
(10) Pub. No.: US 2008/0184446 P1
(43) Pub. Date: Jul. 31, 2008

(54) HELIOTROPIUM PLANT NAMED 'HELIOSIL'

(75) Inventor: Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/651,393

(22) Filed: Jan. 9, 2007

(51) Int. Cl. A01H 5/00

(52) U.S. Cl. PLT/438

(57) ABSTRACT

A distinct Heliotropium plant particularly distinguished by its light lavender color, vigor, good branching, and floriferous characteristics. The new cultivar can be used in 10-12 cm pots without use of growth regulators.

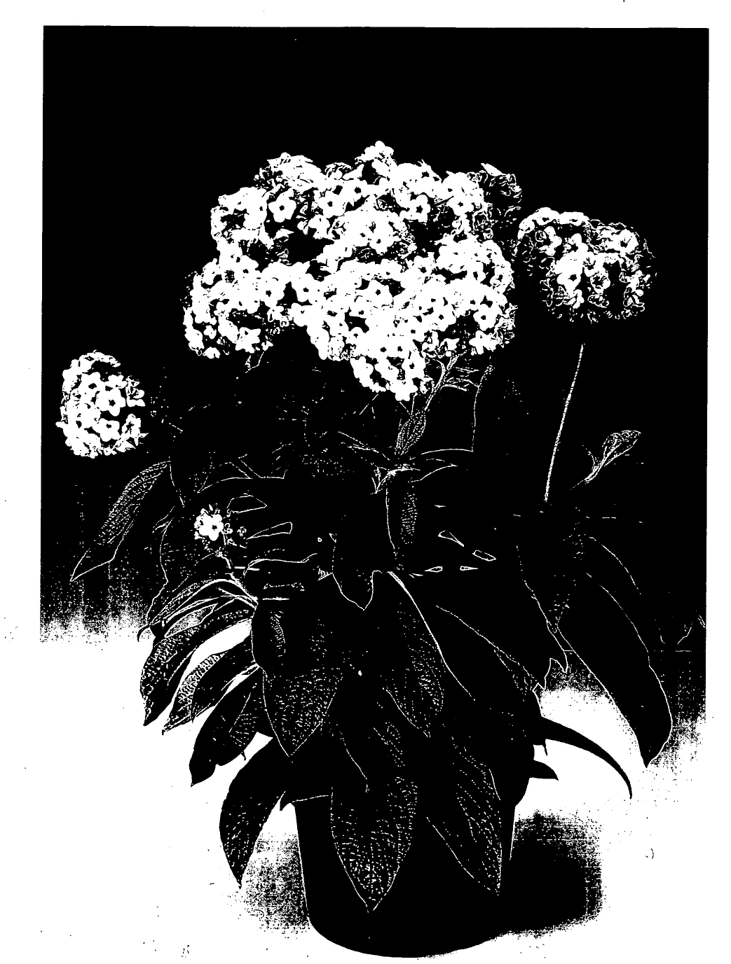

### LATIN NAME OF THE GENUS AND SPECIES OF THE PLANT CLAIMED

Heliotropium arborescens

### VARIETAL DENOMINATION

‘Heliosil’

### BACKGROUND OF THE NEW PLANT

The present invention relates to a new distinct cultivar of Heliotropium, botanically known as Heliotropium arborescens.

The new cultivar is propagated by cuttings resulting from a proprietary Heliotropium arborescens seedling identified as ‘A260-1,’ not patented. Seeds of ‘A260-1,’ resulting from open pollination were harvested in 2001 and the new variety ‘Heliosil’ was selected in 2002 as one of the plants of this population. Breeders's code of the new plant is ‘D3235-1.’

This new plant is an annual. The new cultivar is a product of a planned breeding program conducted in Enkhuizen, The Netherlands. The new cultivar ‘Heliosil’ is a compact variety that flowers throughout the Summer. ‘Heliosil’ was asexually reproduced by cuttings for 4 years now and trialled under different conditions in Europe. It has retained its distinctive characteristics through successive propagations and is firmly fixed. The new variety reproduces true to type in successive generations of asexual reproduction.

### DESCRIPTION OF THE DRAWING

This new Heliotropium plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Heliotropium plant. The data which define these characteristics were collected from asexual reproductions carried out in Enkhuizen, Netherlands. The plant history was taken on 30 week old plants, grown in the open field in Enkhuizen, The Netherlands with day temperatures ranging from 16 and 30° Celsius, and night temperatures ranging from 10 to 16° Celsius.

Color references are primarily to the RHS Color Chart of The Royal Horticultural Society of London.

TABLE 1
DIFFERENCES BETWEEN THE NEW CULTIVAR ‘HELIOSIL’
AND TWO SIMILAR VARIETIES

| | ‘Atlanta’ | | |
|---|---|---|---|
| | (U.S. Plant Patent | Marine GWO | |
| | No. 11,326) | (Not patented) | ‘Heliosil’ |
| Plant height | 25 cm | 60 cm | 35-45 cm |
| Plant width | 40-45 cm | 45 cm | 60 cm |
| Branching | Freely branching | Poor branching | Freely branching |
| Foliage color | RHS 145A | RHS 138A | RHS 145A, old |
| | | | leaves RHS 146A |
| Inflorescence | 9-13 cm | 22 cm | Up to 14 cm |
| width | | | |
| Flower color | Dark blue | Dark blue | Light lavender |
| Flower width | 8 mm | 7 mm | 8.5 mm |

The plant:
   Classification-Botanical.—Heliotropium arborescens.
Parentage:
   Female parent.—Proprietary seedling of Heliotropium arborescens ‘A260-1,’ not patented.
   Male parent.—Unknown variety or seedling of Heliotropium Arborescens.
Propagation:
   Type cuttings.—Terminal cuttings.
   Root description.—Fibrous.
   Rooting habit.—Freely branching.
   Time to initiate roots.—9-12 days at air temperature of 21° C.
   Time to develop roots.—14-18 days at air temperature of 17° C.
Plant description:
   Growth habit.—Upright.
   Plant.—Height: 35-45 cm Width: 60 cm.
   Strength.—Resistant to hot and cold weather.
   Branching character.—Freely branching without pinching.
   Number of branches per plant.—1-3 branches at base of plant ±approximately 13 lateral branches.
   Crop time.—8 weeks.
The stem:
   Diameter.—4.5 mm, up to 15 mm.
   Shape.—Cylindrical.
   Length of.—Plant stem: Up to 45 cm Lateral branch: Up to 45 cm Internode: Up to 22 mm, depending on light conditions.
   Pubescence.—Densely, short hairs.
   Color.—Young stem: RHS 147B Older stem: With anthocyan pigmentation; lighter than RHS N199B.
The foliage:
   Arrangement.—Alternate.
   Number of leaves.—Up to 25.
   Shape of leaf.—Elliptic.
   Fragrance.—Light scent of hay.
   Leaf.—Apex: Acute Base: Entire Margin: Laciniate.
   Texture.—Densely covered on lower and upper side with soft, short hairs.
   Leaf.—Length: Up to 11 cm on mature plant Width: Up to 4 cm.
   Color.—Upper side: Young leaf RHS 145A, old leaf RHS 146A Lower side: Young leaf RHS 147C, old leaf RHS 148B.
   Petiole.—Length: 12 mm Color: In young and old leaf: RHS N144D Diameter: 2 mm Texture: Densely pubescent with short, soft hairs.
   Venation.—Shape: Pinnate Color: Upper and lower surface: RHS N144D.
Inflorescence:
   Flowering season.—Continuous and free flowering throughout spring Summer and Fall.
   Type.—Terminal compound cymeses.
   Height.—Up to 4 cm.
   Width.—2.5 cm when first flowers open Up to 14 cm when flowering is finished.
   Nr of open flowers per inflorescence.—Up to 75.
   Peduncle.—Strength: Strong Aspect: At an angle of 45° to the stem Length: 1-4 cm Diameter: 1.5 cm Texture: Densely pubescent Colour: RHS 197A.
   Fragrance.—Strong.
   Lastingness of inflorescence on plant.—4 weeks.
Flower:
   Shape.—Funnel shaped.
   Length.—6 mm.
   Width (of corolla).—8.5 mm.
   Nr of petals.—5, fused at base, forming tube.
   Apex.—Rounded.
   Margin.—Entire.
   Texture.—Upper side: Smooth Lower side: Smooth, occasionally very short hairs, more hairs on veins.
   Color.—Upper side: young flower: RHS N87D, old flower: RHS 85D, base RHS 150D Lower side: young flower: RHS 85D, old flower: RHS 85D.
   Corolla tube.—Length: 4 mm Diameter at base: 1 mm Diameter at tube opening: 1 mm Texture outer surface: Some pubescence Texture inner surface: Smooth Color of outer surface: RHS 86A Color of inner surface: RHS N199A.
   Sepals.—Number: 5, fused at base Shape: Deltoid Length: 3 mm Width: ±0.5 mm Apex: Acute to acuminate Margin: Entire Texture: Upper and lower surface densely pubescent with long soft hairs. Color: RHS 147A.
   Bud.—Shape: Tubular Length: 4 mm Diameter: 1 mm Colour: RHS 85C.
Androecium:
   Anther.—Color: RHS N199D Length: 1.5 mm Shape: Ligulate.
   Stamen.—Number: 5, fused to inside of tube Length 0.3 mm.
   Pollen color.—RHS 155A.
   Amount of pollen.—Abundant.
Gynoecium:
   Pistil.—Number: One Length: 2.5 mm.
   Style.—Length: 0.4 mm Color: RHS 154C.
   Ovary.—Number: One per pistil Size: 0.4 mm diameter, 1.1 mm long Color: RHS 143C.
   Stigma.—Shape: Capitate Color: RHS 143C, top RHS 154B.
Seed:
   Production.—Yes.
   Size.—Length: 1-1.5 mm Diameter: 1 mm.
   Color.—RHS 200A.
   Shape.—Ovoid.
Disease resistance and tolerance for weather conditions: Good tolerance to heat. No specific susceptibility for pests or diseases has been observed to date.

What is claimed is:

1. A new and distinct variety of Heliotropium plant, substantially as illustrated and described herein.
